(19) United States
(12) Patent Application Publication
(10) Pub. No.: US 2024/0382579 A1
(43) Pub. Date: Nov. 21, 2024

(54) METHOD OF PRODUCING A FOOT AND MOUTH DISEASE VIRUS VIRUS-LIKE PARTICLE

(71) Applicant: Intervet Inc., Rahway, NJ (US)

(72) Inventors: Erwin Van Den Born, Wageningen (NL); Carina Leifeld, Rheinberg (DE); Amaya Serrano Garcia, Wageningen (NL); Holger Honemann, Mönchengladbach (DE); Alexandra Jimenez Melsio, Barcelona (ES); Kimberly Pietersz, Almere (NL)

(73) Assignee: Intervet Inc., Rahway, NJ (US)

(21) Appl. No.: 18/683,835

(22) PCT Filed: Aug. 19, 2022
(86) PCT No.: PCT/EP2022/073144

§ 371 Date: Feb. 15, 2024

(30) Foreign Application Priority Data
Aug. 20, 2021 (EP) 21192317.2

(51) Int. Cl. A61K 39/135; A61K 39/00; A61P 37/04; C07K 14/005; C12N 15/86

(52) U.S. Cl. CPC A61K 39/135; A61P 37/04; C07K 14/005; C12N 15/86; A61K 2039/5258; C12N 2710/14043; C12N 2770/32123; C12N 2770/32134; C12N 2770/32152; C12N 2770/32171

(57) ABSTRACT

The invention concerns a method of producing a foot and mouth disease virus (FMDV) virus-like particle (VLP) in a baculovirus expression system, the method comprising the steps of (i) infecting an insect cell with a baculovirus expression vector, (ii) culturing the insect cell in cell culture medium for 4 days or more post infection, (iii) separating the insect cells from the cell culture to obtain cell-free cell culture medium, and (iv) harvesting the FMDV VLP from the cell-free cell culture medium. The invention further relates to a vaccine for use in the protection of a subject against an infection with FMDV, the vaccine being obtainable by the method of the invention.

The present invention relates to the fields of veterinary medicine and virology. The invention particularly concerns a method of producing a foot and mouth disease virus (FMDV) virus-like particle (VLP) in a baculovirus expression system, the method comprising the steps of (i) infecting an insect cell with a baculovirus expression vector, (ii) culturing the insect cell in cell culture medium for 4 days or more post infection, (iii) separating the insect cells from the cell culture to obtain cell-free cell culture medium (also referred to herein as supernatant), and (iv) harvesting the FMDV VLP from the cell-free cell culture medium. The invention further relates to a vaccine for use in the protection of a subject against an infection with FMDV, the vaccine being obtainable by the method of the invention.

### BACKGROUND OF THE INVENTION

Foot-and-mouth disease (FMD) is a highly contagious, acute viral disease of cloven-hoofed, domesticated and wild animals. It is classified as a transboundary animal disease by the Food and Agriculture Organisation of the United Nations (FAO). It is also a notifiable disease. Foot-and-mouth disease is endemic in large parts of Africa, South America, The Middle East and Asia and is, globally, the most economically important infectious disease of livestock, affecting cattle, pigs, sheep, goats and other artiodactyl species like buffalo and deer. FMD was once distributed worldwide but has been eradicated in some regions, including North America and Western Europe. In endemic countries, FMD places economic constraints on the international livestock trade and can be easily reintroduced into disease-free areas unless strict precautions are in place. FMD impacts on the whole livestock industry with loss of income for local farmers.

Current vaccines are made of inactivated virus. Before the virus is inactivated, live FMD virus is produced in high containment facilities, limiting FMD vaccine production. The building costs and maintenance costs of such a facility are higher than that of a conventional facility and due to the limitations that containment brings operating costs are higher as well.

Effective vaccination against FMD requires the presence of intact FMDV capsids (also known as 146S particles) rather than the capsid building blocks that have been proven to be insufficiently immunogenic (Doel and Chong, 1982, Archives of Virology). The inactivated FMD viruses are fragile structures that at acidic pH or at elevated temperatures easily fall apart in the capsid building blocks. Hence, a cold chain is required to deliver effective FMD vaccines to livestock keepers. There is consequently a huge undersupply of vaccine globally, especially in Africa. Therefore, a new vaccine technology for commercial FMD vaccines that can overcome many of the drawbacks of the current classic inactivated virus vaccines is needed.

A new vaccine technology for commercial FMD vaccines that can overcome many of the drawbacks of the current inactivated virus vaccines is needed.

The virus-like particle (VLP) technology is currently considered one of the few technologies with the potential to be a viable alternative to conventional inactivated vaccines. The benefits of the VLP technology as compared to the current technology are for example higher product stability, greater flexibility in production location (low-containment production), and quicker responses to outbreaks of new strains. VLP-based vaccines are designed as marker vaccines which relieves the necessity of implementing production steps to remove non-structural proteins.

The FMDV genome encodes a single open reading frame (ORF) that produces a precursor polyprotein that is processed into twelve mature viral proteins. FIG. 1 (from: Balinda et al. Virology Journal 2010, 7:199). The P1 polyprotein intermediate is comprised of four capsid structural proteins, VP1-VP4, sited immediately upstream of the 2A protein which causes non-proteolytic separation of the P1 and P2 polyproteins during translation to release P1-2A from P2. The P1-2A polyprotein is subsequently processed by the FMDV 3C protease into 2A, VP0 (also known as 1AB), VP3 (1C), and VP1 (1D). It is believed that the VP0 protein separates into VP4 and VP2 during encapsulation. FMDV virions are formed by self-assembly from the processed virus structural proteins.

VLPs for use in VLP-based vaccines can be produced by recombinantly expressing FMDV precursor proteins in suitable host cells in analogy to the self-assembly of FMDV virions. The baculovirus expression vector platform is currently used as one of the preferred platforms for the production of VLPs. For example, recombinant expression can be performed in the baculovirus expression system using a modified 3C protease that is less toxic to the insect cells (Porta et al (2013) J Virol Methods). VLPs self-assemble from the processed virus structural proteins, VP0, VP3 and VP1, which are released from the structural protein precursor P1-2A by the action of the virus-encoded 3C protease. Intermediate and non-toxic activity of the 3C enzyme in a P1-2A-3C cassette allows expression and processing of the P1-2A precursor into the structural proteins which assemble into empty capsids. The thermostability and resistance to low pH of VLPs can be improved by the introduction of covalent links between the capsid proteins, such as cysteine bridges (WO2002/000251), or by the introduction of other rationally designed mutations (Porta et al. (2013) PLOS Pathog).

However, the relatively low expression levels of FMDV VLPs provided by the baculovirus expression platform limits the development of a VLP-based FMD vaccine.

Proteins that are produced in the baculovirus expression system usually end up inside the insect cells, unless the proteins contain a signal sequence that targets them to the extracellular environment. Recombinant proteins that are trapped inside insect cells can be released by cell disruption techniques known in the art. The obtained cell lysate contains all the cellular components and debris, and often requires laborious purification to obtain the recombinant protein in a purer form. Further, cell disruption techniques also release a lot of unwanted cellular proteins, such as proteases, which can degrade the desired proteins, thereby reducing protein yield and quality. Therefore, targeting sequences are often deliberately engineered into the protein sequences if the protein of interest should be targeted to the cell culture medium from which it can be easily harvested. This is beneficial because the cell culture medium is much cleaner than cell lysates. In addition, we have found that FMDV VLPs purified from lysed insect cells have only moderate thermostability and are produced at relatively low yields, especially for certain serotypes.

Thus, there is a need in the art for improved methods for producing FMDV VLPs in insect cells that can be easily obtained in high yield and without the need for laborious purification.

### SUMMARY OF THE INVENTION

In the present invention, it has surprisingly been found that FMDV VLPs, although not engineered with signal sequences, were transported to the cell culture medium. This seems to be an active process as it was observed that VLPs are getting abundant in the cell culture medium before cells burst open as a consequence of the baculovirus infection. The benefit of harvesting from the cell culture medium is that crude vaccine antigen is cleaner than cell lysates, and the overall yield of VLPs is improved, because more VLPs per ml culture can be obtained.

Remarkably, it could be observed that VLPs seem to mature while they move to the extracellular matrix. This might explain the surprising observation in the present invention that VLPs derived from the cell culture medium are more stable than the ones derived from cell lysates.

Thus, in a first aspect the present invention provides a method of producing a foot and mouth disease virus (FMDV) virus-like particle (VLP) in a baculovirus expression system, the method comprising the steps of:
(i) infecting an insect cell with a baculovirus expression vector, wherein the insect cell is capable of recombinantly producing the FMDV VLP,
(ii) culturing the insect cell in cell culture medium under conditions in which the insect cell produces the FMDV VLP, wherein culturing is performed for 4 days or more post infection,
(iii) separating the insect cells from the cell culture to obtain cell-free cell culture medium (also referred to herein as supernatant),
(iv) harvesting the FMDV VLP produced by the insect cells from the cell-free cell culture medium.

In a second aspect, the invention, provides a vaccine for use in the protection of a subject against an infection with FMDV, the vaccine being obtainable by the method of the present invention.

In a third aspect, the invention provides a method of protecting a subject against an infection with FMDV, which comprises the step of producing an FMDV VLP by the method of the present invention, incorporating the VLP into a vaccine by addition of a pharmaceutically acceptable carrier, and administering the vaccine to the subject.

### DETAILED DESCRIPTION OF THE INVENTION

### Definition of Terms

A virus “capsid” is commonly understood in the art as the protein shell of a virus, typically enclosing its genetic material.

A “capsid precursor protein” is a structural protein, which takes part in the formation of a virus capsid or of a building block thereof. FMDV capsid precursor proteins typically comprise the structural protein P1. Since the protein P1 is processed by the FMDV 3C protease (3Cpro) into the mature VP0, VP3, and VP1 proteins, the P1 protein may also be referred to as polyprotein or proprotein. In the context of the present invention, the FMDV capsid precursor protein typically comprises at least P1 including the proteins VP1, VP2, VP3 and VP4. Alternatively, the FMDV capsid precursor protein may comprise one or more of the proteins VP1, VP2, VP3 and VP4. The FMDV capsid precursor protein may also comprise the protein VP0 comprising the proteins VP2 and VP4. Most preferably, the FMDV capsid precursor protein at least comprises the P1 and 2A proteins (also referred to herein as P1-2A capsid precursor).

A “virus-like particle” (VLP), which may also be referred to in the art as “empty capsid”, is an entity which comprises the protein shell of a virus but lacks the RNA or DNA genome. A VLP should be antigenic and immunogenic in the same way as the wild-type virus because it retains the same structural epitopes, but it should produce no infection, due to the lack of the virus genome. An FMDV VLP is typically formed from the P1-2A capsid precursor. As described above, the 2A protease cleaves itself at its C terminus to release P1-2A from P2. Processing of the P1-2A capsid precursor is affected by the 3C protease to produce 2A and the capsid proteins VP0, VP3 and VP1. The VLP is formed by self-assembly from these capsid proteins.

VLPs may also be produced in the baculovirus expression system of the present invention using a modified 3C protease that is less toxic to the insect cells (Porta et al. (2013) J Virol Methods). Intermediate and non-toxic activity of the 3C enzyme in a P1-2A-3C expression cassette allows recombinant expression and processing of the P1-2A precursor into the structural proteins, VP0, VP1, and VP3, which assemble into VLPs. The production of VLPs may be investigated or verified using techniques known in the art such as sucrose density centrifugation or electron microscopy. Monoclonal antibodies specific for conformational epitopes on the wild-type virus may be used to investigate whether the structure and antigenicity of the empty capsid is retained.

The term “vaccine” as used herein refers to a preparation which, when administered to a subject, induces or stimulates a protective immune response. A vaccine can render an organism immune to a particular disease.

To “protect an animal against an infection with FMDV” means aiding in preventing, ameliorating or curing a pathogenic infection with FMDV. or aiding in preventing, ameliorating or curing a disorder arising from that infection, for example to prevent or reduce one or more clinical signs resulting from a post treatment (i.e. post vaccination) infection with FMDV.

The term “prevention” or “preventing” is intended to refer to averting, delaying, impeding or hindering the FMDV infection by a prophylactic treatment. The vaccine may, for example, prevent or reduce the likelihood of an infectious FMDV entering a host cell.

The term “nucleic acid sequence” includes an RNA or DNA sequence. It may be single or double stranded. It may, for example, be genomic, recombinant, mRNA or cDNA.

An “expression vector” (syn. “expression construct”), is usually a plasmid or virus designed for recombinant gene expression in cells. The vector is used to introduce a specific gene into a target cell, and can commandeer the cell's mechanism for protein synthesis to produce the protein of interest (POI) encoded by the gene. In order to express the recombinant gene to produce the POI, the expression vector typically comprises at least a promotor to drive the expression of the gene of interest (GOI) and may further comprise one or more translational enhancers to increase the yield of the POI.

A “baculovirus expression vector” is an expression vector based on a baculovirus, which is used for recombinant gene expression in a host cell, such as an insect cell. Baculovirus expression systems are established in the art and are commercially available, such as the Bac-to-Bac expression system (ThermoFisher Scientific, Germany). In these baculovirus expression systems, the naturally occurring polyhedrin gene within the wild-type baculovirus genome is typically replaced with a recombinant gene or cDNA. These genes are commonly under the control of the polyhedrin or p10 baculovirus promoters.

The most common baculovirus used for gene expression is Autographa californica nucleopolyhedrovirus (AcNPV). AcNPV has a large (130 kb), circular, double-stranded DNA genome. The GOI is cloned into a transfer vector containing a baculovirus promoter flanked by baculovirus DNA derived from a nonessential locus, such as the polyhedrin gene. The recombinant baculovirus containing the GOI is produced by homologous recombination in insect cells between the transfer vector and the genome of the parent virus (such as AcNPV).

A “translational enhancer” is a nucleotide sequence forming an element, which can promote translation and, thereby, increase protein production. Typically, a translational enhancer may be found in the 5′ and 3′ untranslated regions (UTRs) of mRNAs. In particular, nucleotides in the 5′-UTR immediately upstream of the initiating ATG codon of the GOI may have a profound effect on the level of translation initiation.

### Baculovirus Expression System

In the method of the present invention, the FMDV VLP is produced in a baculovirus expression vector system (BEVS) using a baculovirus expression vector.

The baculovirus expression vector can be any baculovirus expression vector capable of recombinantly expressing an FMDV capsid precursor protein under control of a promoter. The promoter is not particularly limited but may be any promoter capable of recombinantly expressing the FMDV capsid precursor protein in a baculovirus expression system. Preferred promoters for use in the baculovirus expression system of the present invention are the polyhedrin (polh) promoter (described in: Ayres M.D. et al. (1994) Virology, Vol. 2020, p. 586-605) and the p10 promoter (described in: Knebel D. et al. (1985) EMBO J. Vol. 4(5), 1301-1306) of AcNPV. Another preferred promoter is the promoter of the orf46 viral gene of S. exigua nucleopolyhedrovirus (SeNPV) (described in M. Martinez-Solis et al. (2016) PeerJ, DOI 10.7717/peerj.2183).

The expression vector may further comprise one or more translational enhancers, which enhance the recombinant expression of the FMDV capsid precursor protein. For example, the baculovirus expression vector may comprise the two translational enhancers Syn21 and p10UTR as described in EP 20 203 373, incorporated herewith by reference in its entirety.

Baculovirus expression vectors for use in baculovirus expression systems for the recombinant expression of proteins are commercially available and are extensively used in the art for the production of proteins and virus-like particles. The systems may encompass, for example, one or more transfer plasmids used to transform cells, such as E. coli cells or insect cells, in which the baculovirus expression vector is propagated. Commercially available baculovirus expression vectors include, but are not limited to, Top-Bac® vector (ALGENEX, Spain), pFastBac® vector (Thermo Fisher Scientific, Germany), flashBAC® vector (Oxford Expression Technologies Ltd, UK) and BestBac® vector (EXPRESSION SYSTEMS, CA).

The baculovirus expression vector used in the method of the present invention thus may contain an expression cassette comprising the nucleic acid sequence encoding the FMDV capsid precursor protein, which is expressed in the insect cell under control of a functional promoter, and preferably including one or more translational enhancers and/or other cis-acting elements.

The nucleic acid sequence encoding the FMDV capsid precursor protein is not particular limited to a certain strain and may be of any FMDV strain belonging to serotype A, O, Asia1, SAT1, SAT2, SAT3 or C. In a particularly preferred embodiment, the FMDV capsid precursor protein is from the A or O serotype.

In the method of the present invention, the FMDV capsid precursor protein may comprise all elements necessary for the processing and assembly of VLPs. Hence, the FMDV capsid precursor protein typically comprises at least the capsid precursor P1 and preferably further comprises the 2A peptide. The 2A peptide is able to release P1-2A from any protein sequence downstream of its C terminus.

In a further preferred embodiment, the baculovirus expression vector further comprises a nucleic acid sequence encoding a protease capable of cleaving an FMDV capsid precursor protein. The protease may be any protease capable of cleaving the FMDV capsid precursor protein as a step in the production and assembly of FMDV VLP. As mentioned above, for FMDV, proteolytic processing of the precursor P1 into VP0 (VP2 plus VP4), VP3 and VP1 occurs by means of the viral 3C protease or its precursor 3CD. Hence, the protease is preferably the 3C protease of FMDV. The sequence of FMDV wild-type 3C protease from an FMDV type A strain is described in the art and is disclosed in WO 2011/048353, which is hereby incorporated by reference in its entirety. The 3C protease may also be a functional derivative including one or more mutations, which reduce its proteolytic activity, for example a mutation at Cysteine 142.

The capsid precursor protein may be P1, which is cleaved by the 3C protease into VP0, VP3 and VP1. Most preferably, the baculovirus expression system expresses a P1-2A-3C cassette, i.e. it simultaneously expresses the coding regions for the proteins P1, 2A and 3C. Expression of the 3C enzyme in a P1-2A-3C cassette allows expression and processing of the P1-2A precursor into the structural proteins which assemble into VLPs. The capsid precursor protein and the protease may be expressed under control of individual promotors or under control of the same promoter. Further alternatively, the capsid precursor proteins required for the assembly of FMDV VLPs may be split up into multiple expression units and expressed separately, for example by recombinantly producing VP1, VP2, VP3 and VP4, or recombinantly producing VP0, VP1 and VP3. In this alternative embodiment, a proteolytic cleavage of a capsid precursor protein by a 3C protease may not be necessary.

Cleavage of the capsid precursor protein or VLP may be analysed using techniques known in the art. For example, extracts from baculovirus-infected host cells may be analysed by gel-electrophoresis and the separated proteins transferred onto a nitrocellulose membrane for Western blotting. Western blotting with protein-specific antibodies should reveal the degree of protease-mediated cleavage. For example, for FMDV, the unprocessed capsid precursor protein (P1-2A) would appear as a band of around 81 kDa, and cleavage may produce VP3-VP1 (˜47 kDa), VP0 (˜33 kDa), VP2 (˜22 kDa), VP3(˜24 kDa) and/or VP1 (˜24 kDa).

### Method of Producing Virus Like Particles

The method of the present invention includes the culturing of the host cell, which in the invention is an insect cell, under conditions suitable for the cell to express the capsid precursor protein from the baculovirus expression vector in order to produce VLPs. The term “the insect cell is capable of recombinantly producing the FMDV VLP” thus means that the insect cell can be used as a host cell for the production of recombinant capsid precursor proteins, which assemble into VLPs.

The first step of the method of the invention comprises infecting an insect cell with the baculovirus expression vector (step (i) of the method of the invention). The insect cell my be any insect cell, which is capable of producing FMDV VLPs in cell culture. In particular, the insect cell may be a Sf9 cell (a clonal isolate of Spodoptera frugiperda Sf21 cells), Sf21 cell, High-Five® (BTI-TN-5B1-4) cell or a Tni cell (ovarian cells isolated from Trichoplusia ni). Most preferably, the host cell is a Tni cell, or a Tni-derived cell line, such as a Tnao38 cell.

Methods of infecting an insect cell with a baculovirus expression vector for the recombinant expression of proteins are well known to the skilled person.

In the method of the invention, culturing of the insect cell is performed in cell culture medium (step (ii) of the method of the invention), such as a suspension cell culture in serum free medium.

Cell culture of infected insect cells under conditions under which the insect cell produces the FMDV VLP is established in the art and can be performed, for example, as described in (Porta et al., 2013, J. Virol. Methods, vol. 187, p. 406; A.C. Mignaqui et al., 2019, Critical Reviews in Biotechnology, vol. 39(3), p. 306-320). The general procedures for recombinant protein expression using the BEVS in insect cell cultures are well known in the art and are described, for example, in the “Guide to Baculovirus Expression Vector Systems (BEVS) and Insect Cell Culture Techniques”, Invitrogen®, Instruction Manual; L. King, The Baculovirus Expression System, A laboratory guide; Springer, 1992; Baculovirus and Insect Cell Expression Protocols, Humana Press, D.W. Murhammer (ed.) 2007; Baculovirus Expression Vectors: A Laboratory Manual, Oxford University Press, D.R. O'Reilly, 1993.

Traditionally, Grace's Supplemented (TNM-FH) medium has been the medium of choice for insect cell culture. However, other serum/hemolymph-dependent and serum-free formulations have evolved since Grace's medium was introduced. The optimal range for growth and infection of most cultured insect cells is typically 25° C. to 30° C. at a pH range of 6.0 to 6.4.

In the method of the present invention culturing of the infected cells is performed for 4 or more days post infection (dpi). It could surprisingly be observed in the present invention that on day 4 and 5 post baculovirus infection the VP0 protein was present in the cell culture medium and was absent at day 6 and 7 post infection. Concomitant with the disappearance of VP0, the VP2 protein appeared in the cell culture. Hence, it could be shown that the VP0 protein in the culture medium was cleaved into the VP2 and VP4 proteins. The cleavage of VP0 into VP2 and VP4 is thought to occur at the final stage of virus particle maturation (Curry et al., 1997, J. Virol. 71:9743-9752).

Hence, even though the recombinant capsid precursor protein produced by the insect cell may lack a signal sequence, the VLPs formed from the recombinant capsid precursor protein are released by the cell into the cell culture medium. In addition, it was surprisingly observed that cell culture medium is a good source of vaccine antigen, because it contains matured VLPs, in contrast to the cells, which do not contain significant amounts of matured VLPs.

Hence, in a particularly preferred embodiment of the invention, culturing is performed for five or more days post infection, such as five, six or seven days, preferably for five or six days, most preferably for five days. Although higher yields are obtained when culturing for more than five days, the extra culturing time needed is disadvantageous from a costs point of view. Five days is found to be optimal.

After culturing, the insect cells are separated from the cell culture to obtain cell-free cell culture medium (also referred to as supernatant; step (iii) of the method of the invention). The term “supernatant” thus relates to the cell culture from which the insect cells have been removed.

In the method of the present invention, the VLPs are obtained from the supernatant, only. Hence, the cells are removed from the cell culture to obtain a cell culture medium, also referred to herein as supernatant, which is substantially free of insect cells. “Substantially free” of insect cells means that only residual cells may be present, which are insignificant for producing VLPs in the present invention. Most preferably, the supernatant does not contain any residual cells.

Conventional techniques for separation of the cells from small—or large—scale cell cultures are well known in the art and include one or more of membrane filtration, such as ultrafiltration, centrifugation and sedimentation.

The VLPs in the supernatant can be concentrated by dialysis, membrane filtration, or precipitation followed by centrifugation.

In step (iv) of the method of the present invention, the FMDV VLPs produced by the insect cells are harvested from the cell culture medium. Harvesting may include the separation of the VLPs from the culture medium and, if necessary, further purification of the VLPs. Harvesting can be performed by precipitation of the VLPs, for example with polyethylene glycol (PEG). Chromatographic techniques such as affinity chromatography or ion exchange chromatography can also be used to purify and concentrate the VLPs. Harvesting may also include ultrafiltration to concentrate the VLPs in the cell culture medium or diafiltration to concentrate the VLPs and replace the cell culture medium with a liquid or buffer of choice. If the concentration and purify of the VLPs in the cell culture medium is high enough for vaccine production, step (iv) of the present invention may not involve any purification steps.

During the harvesting, concentration and/or purification procedures, the presence of protease inhibitors may diminish undesirable proteolytic activity.

### Vaccines and Production Thereof

As described above, the preferred utility of the embodiments of the present invention is in veterinary medical use, in particular for vaccination against FMD. The present invention thus further relates to the production of FMDV VLPs, which are used in the production of a vaccine.

In particular, the VLPs harvested from the cell culture medium in step (iv) of the method according to the invention may be used as antigen for vaccination of subjects. Preferably, the VLPs are incorporated into a composition comprising the VLPs and one or more pharmaceutically acceptable carriers.

The present invention thus also provides a method for the production of a vaccine, which comprises the step of producing FMDV VLPs by a method as described above and incorporating the FMDV VLPs in a vaccine, such as by the addition of a pharmaceutically acceptable carrier.

Pharmaceutically acceptable carriers are well-known in the art. Merely as an example; such a carrier can be as simple as sterile water or a buffer solution such as PBS. The vaccine may comprise a single carrier or a combination of two or more carriers. The vaccine may also comprise one or more pharmaceutically acceptable diluents, adjuvants and/or excipients. The vaccine may also comprise, or be capable of expressing, another active agent, for example one which may stimulate early protection prior to the VLP-induced adaptive immune response. The agent may be an antiviral agent, such as type I interferon. Alternatively, or in addition, the agent may be granulocyte-macrophage colony-stimulating factor (GM-CSF).

The vaccine may be used therapeutically, to treat an existing FMDV infection (especially in herds or regions where the virus is endemic), but preferably is used prophylactically, to block or reduce the likelihood of FMDV infection and/or prevent or reduce the likelihood of spreading the disease.

Many commercially available FMD vaccines are multivalent to provide protection against the different FMD serotypes. By the same token, the vaccine of the present invention may comprise a plurality of different VLPs, each directed at a different serotype and/or different subtypes within a given serotype.

Thus, in a further preferred embodiment, the method of the invention further comprises the step (v) of incorporating the FMDV VLPs into a vaccine by addition of a pharmaceutically acceptable carrier.

The vaccine obtained by the method as described above may be used in the protection of a subject against an infection with FMDV.

The present invention also provides a method of protecting a subject against an infection with FMDV by administration of an effective amount of a vaccine of the present invention. A method of protecting a subject against an infection with FMDV comprises the step of producing an FMDV VLP by a method as described above, incorporating the VLP into a vaccine by addition of a pharmaceutically acceptable carrier, and administering the vaccine to the subject.

For FMD the subject may be a cloven-hoofed animal. FMD susceptible animals include cattle, sheep, pigs, and goats among farm stock, as well as camelids (camels, llamas, alpacas, guanaco and vicuna). Some wild animals such as hedgehogs, coypu, and any wild cloven-footed animals such as deer and zoo animals including elephants are also susceptible to FMD.

### Administration

The present invention contemplates at least one administration to an animal of an efficient amount of the vaccine according to the invention. A vaccine can be administered in any art-known method, including any local or systemic method of administration. Administration can be performed e.g. by administering the antigens into muscle tissue (intramuscular, IM), into the dermis (intradermal, ID), underneath the skin (subcutaneous, SC), underneath the mucosa (submucosal, SM), in the veins (intravenous, IV), into the body cavity (intraperitoneal, IP), orally, anally etc. For the current vaccine IM, ID and SC administration are preferred.

### EXAMPLES

The invention will be further described by way of the following non-limiting examples, which are meant to serve to assist one of ordinary skill in the art in carrying out the invention.

### BRIEF DESCRIPTION OF FIGURES

FIG. 1: Schematic representation of the FMDV genome encoding a single open reading frame (ORF) that produces a precursor polyprotein that is processed into twelve mature viral proteins.

FIG. 2: Result of the time course experiment with O/TUR/5/2009 VLPs that were harvested at 4, 5, 6, or 7 dpi. FMD proteins in the cells (C) or cell culture supernatant(S) were visualized by Western blotting.

FIG. 3: Quantification by ELISA of O/TUR/5/2009 protein in the cell culture supernatant harvested at different time points after infection.

FIG. 4: Quantification by ELISA of O/TUR/5/2009 protein in different samples.

FIG. 5: Western blot analysis of fractions derived from a 20-40% sucrose gradient. Bands were visualized with both an anti-VP0 and anti-VP2 antibody. Percent sucrose per fraction is indicated below the blot.

FIG. 6: Western blot analysis of samples derived from cultures harvested at either 4 or 7 dpi. Bands were visualized with a polyclonal cattle serum.

FIG. 7: Percent dissociation of Asial/Shamir/89 VLPs incubated at 56° C. for 20 min.

FIG. 8: Virus neutralizing titers induced after vaccination of cattle with O/TUR/5/2009 VLPs derived from either insect cells or cell culture supernatant.

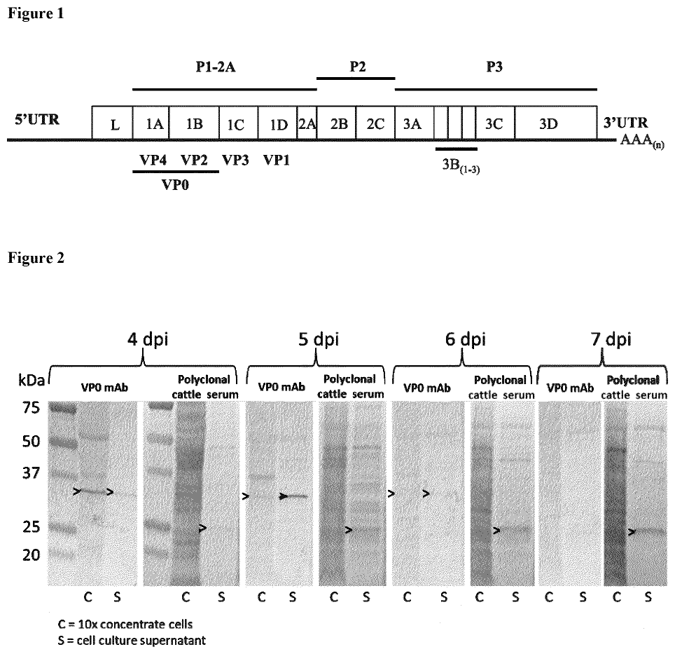

### PREPARATION OF BACULOVIRUS CONSTRUCTS

Cloning of the baculovirus expression constructs was performed by standard cloning procedures well known in the art. The following baculovirus expression constructs were used in the following examples for the recombinant production of VLPs in insect cells:
i) Expression construct O/TUR/5/2009 containing the P1-2A-3Cpro expression cassette of FMDV strain O/TUR/5/2009 not stabilized with any mutation;
ii) Expression construct O/TUR/5/2009-VP2-S93F containing the P1-2A-3Cpro expression cassette of FMDV strain O/TUR/5/2009 stabilized with the VP2-S93F mutation as described in WO 2014/154655 A1;
iii) Expression construct A/IRN/7/2013-VP2-H93F containing the P1-2A-3Cpro expression cassette of FMDV strain A/IRN/7/2013 stabilized with the VP2-H93F mutation as described in WO 2014/154655 A1;
iv) Expression construct SAT2/SAU/6/2000-VP1-T12N-VP4-D53G containing the P1-2A-3Cpro expression cassette of FMDV strain SAT2/SAU/6/2000 stabilized with a mutation in VP1 (T12N) and in VP4 (D53G);
v) Expression construct Asial/Shamir-VP2-S93C containing the P1-2A-3Cpro expression cassette based of FMDV strain Asial/Shamir/89 stabilized with the VP2-S93C mutation as described in WO 2014/154655 A1.

### Example 1

A 100 ml cell culture of Tni cells with a concentration of 3.2×10⁵ cells/ml was infected at MOI=1 with recombinant baculoviruses containing the expression cassette O/TUR/5/2009. After incubation at 27° C. the cells were collected at 4, 5, 6, or 7 days post infection (dpi) by centrifugation and resuspended in 10% of the original cell culture volume, resulting in a 10x concentration factor. No cell lysis method was applied. Cell culture supernatant, i.e. cell culture medium from which the insect cells have been removed by centrifugation, was left untreated. Supernatant and cell samples were analyzed by Western blotting using both the anti-VP0 monoclonal antibody (Loureiro et al., 2018, https://wellcomeopenresearch.org/articles/3-88) and polyclonal cattle serum FMD13.70.445 (MSD Animal Health).

The results in FIG. 2 show that as early as 4 dpi FMDV proteins are detected in the cell culture supernatant. Over time, and most clearly observed on the VP0 Western blot, the amount of FMDV proteins in the cells decreased while the amount of FMDV proteins in the cell culture supernatant increased, showing that the FMDV recombinant proteins are efficiently released from the cell into the culture medium.

The polyclonal serum blot revealed another interesting observation, which is likely linked to capsid maturation. On day 4 and 5 the VP0 protein was present in the cell culture medium and was absent at day 6 and 7 post infection. Concomitant with the disappearance of the VP0 band, a band appears on the polyclonal serum blot that could represent the VP2 protein. If so, the Western blots indicate that the VP0 protein in the culture medium was cleaved into the VP2 and VP4 proteins. The cleavage of VP0 into VP2 and VP4 is thought to occur at the final stage of virus particle maturation (Curry et al., 1997, J. Virol. 71:9743-9752). This is a surprising finding as empty capsids do not contain the RNA genome and usually do not contain cleaved VP0. However, it could indicate that cell culture medium is a good source of vaccine antigen, because it contains matured VLPs, in contrast to the cells.

To quantify the difference in concentration of FMDV protein in the cell culture supernatant an ELISA was performed using the INT-FMA-01-08 monoclonal antibody (MSD Animal Health) which detects both intact capsids (75S/146S) and pentameric building blocks of the capsids (12S). For this, serially diluted samples were incubated for 1h at 37° C. on microtiter plates coated overnight at 4° C. with antibody. After removing the samples and three washes with PBS-Tween, a fixed amount of biotinylated INT-FMA-01-08 was added to plates and incubated for 1h at 37° C. The biotinylated antibody was removed and plates were washed three times with PBS-Tween, after which peroxidase-conjugated streptavidin was added to the plates followed by chromophoric detection.

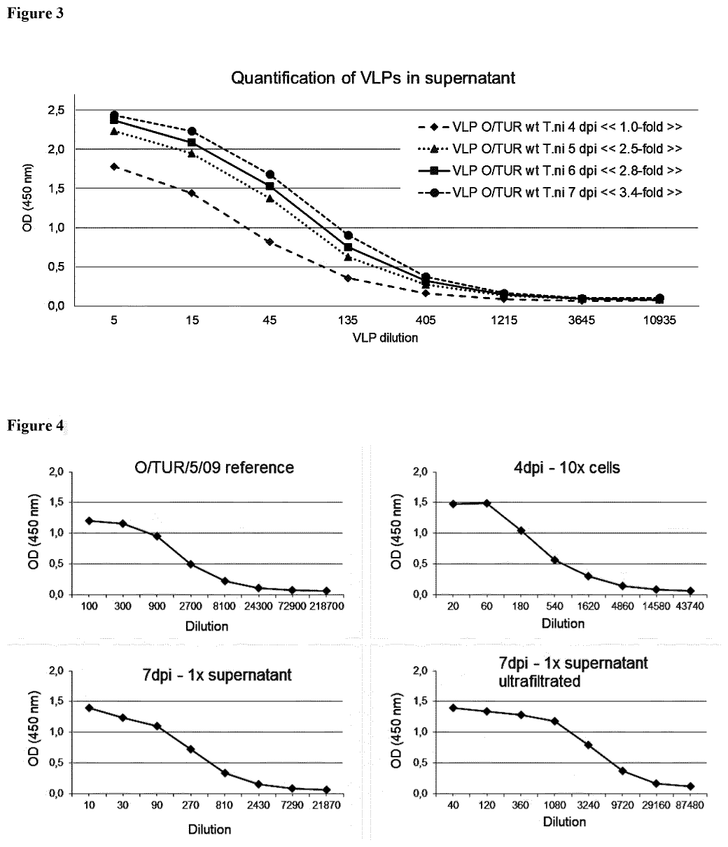

The graph in FIG. 3 is a visual representation of the ELISA results and demonstrates that the amount of VLPs in the cell culture media increased up to 3.4-fold at 7 dpi as compared to that at 4 dpi. By comparing ELISA data of untreated samples with that of samples that were heat treated at 56° C. for 50 minutes to convert the 75S capsids into 12S pentamers, it was estimated that the VLP integrity (i.e. the amount of 75S) in the supernatant for unstabilized wild-type O/Tur/5/2009 was 54%, indicating that indeed intact capsids were released into the cell culture media.

In this Example it is shown that FMDV recombinant proteins are efficiently released from the cells into the culture medium with an increasing amount of VLPs in cell culture medium over time, forming maturated VLPs.

### Example 2

Two 100 ml cell cultures with 3.2×10⁵ cells/ml of Tni cells were infected at MOI=2 with recombinant baculoviruses containing the expression cassette O/TUR/5/2009-VP2-S93F. After incubation at 27° C. cells from one culture were collected at 4 dpi by centrifugation and the cell pellet was subsequently sonicated in 50 mM Tris pH 8.0-100 mM KCl buffer at 10% of the volume of the infection culture. Cell culture supernatant from the second culture was obtained by centrifugation at 7 dpi.

The different time of harvest for each of the fractions was based on the data presented in Example 1 that indicated that the amount of recombinant proteins in cells was highest at 4 dpi while that in cell culture media was highest at 7 dpi. To verify if cell culture supernatant can be concentrated by a simple method, the material was concentrated by applying an ultrafiltration (UF) step using a system with a 100 kDa molecular weight cut-off membrane. To quantify the amount of FMDV recombinant proteins in samples an ELISA was performed using the INT-FMA-01-08 monoclonal antibody as described in Example 1. A reference with a known concentration (in ELISA units/ml or EU/ml) was included in the ELISA to estimate the concentration of the samples.

FIG. 4 shows the individual ELISA graphs, while the obtained values are shown in Table 1. From this data it can be concluded that significantly more FMDV VLPs can be harvested from the cell culture supernatant of the baculovirus expression system as compared to the cells (about 6x), and the supernatant can be concentrated by a 1-step method that can be easily applied in large-scale production.

TABLE 1
Quantification by ELISA of O/TUR/5/2009
protein in different samples

| | | Calculation of |
|---|---|---|
| | Concentration in | concentration in original |
| Sample | sample (EU/ml) | cell culture (EU/ml) |
| 4dpi - cells (10x) | 64 | 6.4 |
| 7dpi - supernatant (1x) | 40 | 40 |
| 7dpi - supernatant, UF | 570 | 32 |
| concentrated (~18x) | | |

In this Example it was shown that more VLPs of an O strain can be harvested from cell culture supernatant than from cells.

### Example 3

Two 100 ml cell cultures with 3.2×10⁵ cells/ml of Tni cells were infected at MOI=1 with recombinant baculoviruses containing the expression cassette O/TUR/5/2009-VP2-S93F. After incubation at 27° C. cells from one culture were collected at 4 dpi by centrifugation and the cell pellet was sonicated in 50 mM Tris pH 8.0-100 mM KCI buffer at 10% of the volume of the infection culture. Cell culture supernatant from the second culture was obtained by centrifugation at 7 dpi. Samples containing lysed cells and supernatant were subjected to zonal gradient centrifugation. The gradient consisted of 20% to 40% sucrose and samples were loaded on top of the gradient prior to centrifugation at 50,000xg for 50 min at 20° C. Fractions of the gradient were analyzed by Western blotting using the anti-VP2 monoclonal antibody F1412SA (Yang et al., 2007, Vet Immunol Immunopathol).

The Western blot analysis shows that VP0 and/or VP2 proteins were detected in the gradient around a sucrose concentration of 35% where 75S particles are to be expected, indicating that in both cells and supernatant intact VLPs are present (FIG. 5). The Western blot analysis also indicates that VLPs in the supernatant have their VP0 partly processed into VP4 and VP2, as indicated by the relatively strong presence of the VP2 band as compared to the VP0 precursor band. This result confirms our earlier observation in Example 1.

In this Example it could be shown that cell culture supernatant contains intact VLPs of an FMDV O strain.

### Example 4

Two 100 ml cell cultures with 3.2×10⁵ cells/ml of Tni cells were infected at MOI=1 with recombinant baculoviruses containing the expression cassette A/IRN/7/2013-VP2-H93F and subsequently incubated at 27° C. One of the two cultures was harvested at 4 dpi and the second was harvested at 7 dpi. The cells and cell culture supernatant fractions were separated by centrifugation. The obtained cell pellet was sonicated in 50 mM Tris pH 8.0-100 mM KCl buffer at 10% of the volume of the infection culture. Cell culture supernatant was left untreated.

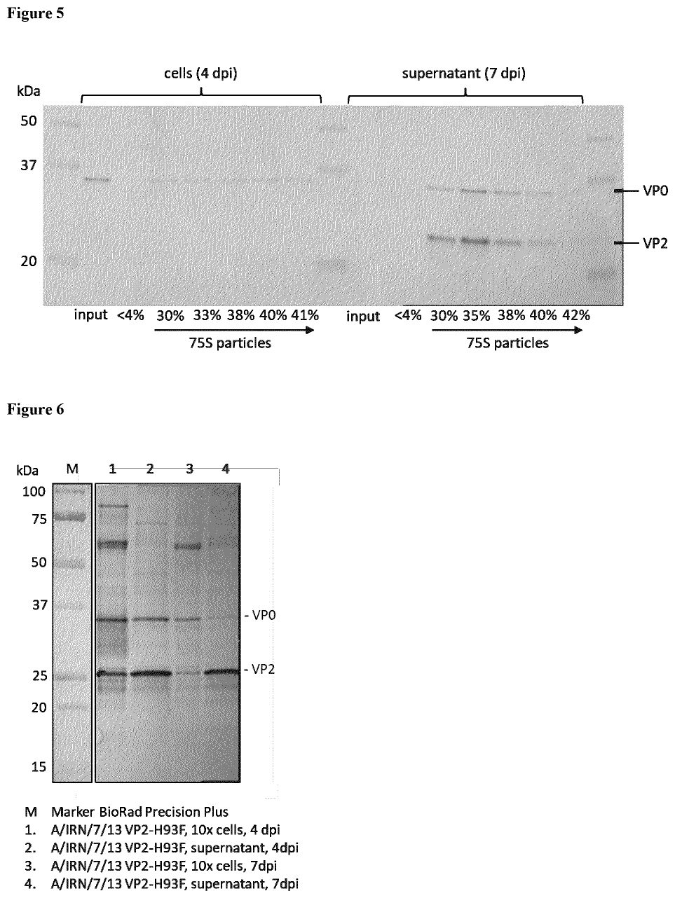

Cell and supernatant samples were analyzed by Western blotting using polyclonal cattle serum FMD13.70.445 (FIG. 6). Visual inspection of the Western blot indicated that the intensity of the VP2 band of the supernatant samples was stronger than that of the cell lysate samples. Because the cell lysates were concentrated 10 fold, it can be concluded that at least 10 times more VP2 protein was present in the extracellular environment at both 4 and 7 days post infection. The Western blot also shows that the cell culture supernatant contained much less P1 polyprotein processing intermediates, and that most of the VP0 protein is cleaved into VP2 (and VP4). This again suggests that mature capsids are predominantly present in cell culture supernatant, as already discussed in Examples 1 and 3.

In this Example it could be shown that more VLPs of an FMDV A strain are in the cell culture supernatant than in cells at both 4 and 7 dpi.

### Example 5

Tnao38 insect cells at a concentration of 2.2×10⁶ cells/ml in a 2-liter bioreactor were infected at MOI=1 with recombinant baculoviruses containing the expression construct SAT2/SAU/6/2000-VP1-T12N-VP4-D53G. After incubation at 28° C. and t 5 dpi cells were collected by centrifugation and the cell pellet was sonicated in 50 mM pH 8.0-100 mM buffer at 5% of the volume of the infection culture. The culture supernatant from the centrifugation step was further concentrated 18.6 fold by ultrafiltration using a 30 kilodalton (kDa) molecular weight cut-off membrane.

The concentration of intact virus-like particles was determined by ELISA using VHH M377F (Harmsen et al., 2017, Front. Immunol.). For this, serially diluted samples were incubated for 1h at room temperature (RT) on microtiter plates coated overnight at 4° C. with M377F. After removing the samples and three washes with PBS-Tween, a fixed amount of a biotinylated M377F was added to plates and incubated for 1h at RT. The biotinylated antibody was removed and plates were washed three times with PBS-Tween, after which peroxidase-conjugated streptavidin was added to the plates followed by chromophoric detection. According to the ELISA, the 20x cell lysate contained 117 EU/ml of intact VLPs, while the concentrated culture supernatant contained 92 EU/ml. Thus, 46% of all SAT2/SAU/6/2000 VLPs are present in the cell culture supernatant at 5 dpi.

As the result, it could be shown that FMDV SAT2 VLPs accumulate in the cell culture supernatant.

### Example 6

Erlenmeyer flasks containing 40 ml of 1·10⁶ Tnao38 insect cells per ml were inoculated with 3 ml of a P1 stock of recombinant baculoviruses containing the expression construct Asia1/Shamir-VP2-S93C. After incubation at 27.5° C. for 4 or 6 dpi the cells were collected by spinning them down for 5 min at 3000 rpm. The resulting cell pellet was resuspended in 50 mM HEPES pH 8.0-100 mM KCl with a volume of 1/10 of the original culture volume and cells were lysed by sonication. The cell culture supernatant was also collected after centrifugation.

The obtained material was heat treated at 56° C. for 20 minutes and the amount of intact VLPs was determined before and after heat treatment by homologous ELISA using the M332F antibody (Harmsen et al., 2017, Front. Immunol. 8:960) according to the method described in Example 5 but with incubation at 37° C. instead of RT.

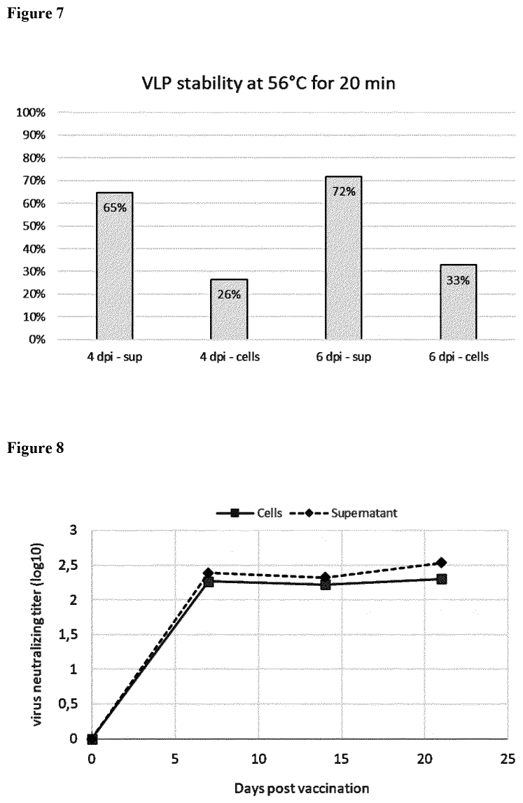

The percentage of capsids that survived the incubation at 56° C. is shown in FIG. 7. The results demonstrate that supernatant-derived VLPs are more thermostable than cell-derived VLPs and a longer culture time (i.e. 6 days instead of 4) seems to improve the thermostability. This observation is not believed to be a result of the stabilizing effect of insect cell culture media on those VLPs, since in additional experiments aimed to measure the effect of cell culture media on VLP thermostability, a stabilizing effect could not be detected. A plausible explanation is that the VLPs in the cell culture supernatant are more matured, because they have been actively transported to the extracellular environment, like the FMDV capsids do in naturally infected cells. In line with the VLP maturation theory (as mentioned in Example 1) is the finding that VLPs become more heat resistant over time: the thermostability of the VLPs harvested at 6 dpi is higher than at 4 dpi.

In this Example it could be demonstrated that thermostability of FMDV VLPs of the Asial/Shamir/89 strain derived from cell culture supernatant is higher compared to VLPs derived from cells.

### Example 7

An animal trial was performed to demonstrate that VLPs derived from the cell culture supernatant are at least as immunogenic as the VLPs derived from the cells. Ten calves, 4-6 months old, were grouped in 2 groups containing 5 calves each. On day 0, calves were vaccinated intramuscularly (IM) with 2 ml of a vaccine formulated with 8 μg of FMDV VLPs of strain O/TUR/5/2009 and the proprietary SVEA-E adjuvant. One group received VLPs derived from the insect cells, while the other group received VLPs derived from the cell culture supernatant. Blood samples were taken at 0, 7, 14, and 21 days post vaccination (dpv). Serum was derived from clothed blood and subsequently tested by virus neutralization assay (VNT) using O/TUR/5/2009.

The O/TUR/5/2009 VP2-S93C VLPs were produced at 30° C. in 2-liter bioreactors containing 2·10⁶ Tnao38 insect cells per ml that were infected at MOI=1. Cell culture supernatant and cells were harvested at 5 dpi by centrifugation at 200xg. VLPs were released by sonication. The concentration of intact VLPs was determined by ELISA using VHH C1 (Wang et al., 2015, BMC Veterinary Research 11:120, DOI 10.1186/s12917-015-0437-2) according to the method described in Example 5 but with incubation at 37° C. instead of RT.

In all animals in both groups high levels of FMDV virus-neutralizing antibodies could be detected at 7 dpv resulting in a group average of 2.26 log₁₀ for the cell group and 2.39 log₁₀ for the cell culture supernatant group (FIG. 8). The titers climbed a bit to 2.30 log₁₀ and 2.53 log₁₀, respectively, on day 21 post vaccination. The results indicate that both sources, cells and cell culture supernatant, yield immunogenic VLPs.

As the result, it could be shown that VLPs derived from cell or cell culture supernatant are both immunogenic.

### Conclusions

It could be shown in the present invention that FMDV recombinant proteins from different strains are efficiently released from the cells into the culture supernatant with an increasing amount of VLPs in cell culture medium over time. The FMDV recombinant proteins form maturated VLPs, which accumulate in the cell culture supernatant. These VLPs derived from cell culture supernatant have higher thermostability compared to VLPs derived from cells. The VLPs derived from cell culture supematant are immunogenic and can be used for the vaccination of subjects providing protection against an infection with FMDV.

1. A method of producing a foot and mouth disease virus (FMDV) virus-like particle (VLP) in a baculovirus expression system, the method comprising:
(i) infecting an insect cell with a baculovirus expression vector, wherein the insect cell is capable of recombinantly producing the FMDV VLP,
(ii) culturing the insect cell in cell culture medium under conditions under which the insect cell produces the FMDV VLP, wherein culturing is performed for 4 days or more post infection,
(iii) separating the insect cells from the cell culture to obtain cell-free cell culture medium,
(iv) harvesting the FMDV VLP produced by the insect cells from the cell-free cell culture medium.

2. The method according to claim 1, wherein the FMDV is of the A serotype.

3. The method according to claim 1, wherein the FMDV is of the O serotype.

4. The method according to claim 1, wherein culturing is performed for five days or more post infection.

5. The method according to claim 1, wherein culturing is performed for five days post infection.

6. The method according to claim 1, wherein the cell culture is separated from the insect cells by one or more of membrane filtration, centrifugation, and sedimentation.

7. The method according to claim 1, wherein the VLPs are concentrated by one or more of dialysis, membrane filtration and precipitation.

8. The method according to claim 1, wherein the baculovirus expression vector comprises a nucleic acid sequence encoding a FMDV capsid precursor protein.

9. The method according to claim 8, wherein the baculovirus expression vector further comprises a nucleic acid sequence encoding a protease capable of cleaving the FMDV capsid precursor protein into one or more capsid proteins.

10. The method according to claim 9, wherein the capsid precursor protein comprises the FMDV capsid precursor P1 and the 2A peptide and the protease is 3C.

11. The method according to claim 1, the method further comprising:
(v) incorporating the FMDV VLP into a vaccine by addition of a pharmaceutically acceptable carrier.

12. A vaccine for use in the protection of a subject against an infection with FMDV, the vaccine being obtainable by a method according to claim 11.

13. A method of protecting a subject against an infection with FMDV, which comprises the step of producing an FMDV VLP by a method according to claim 1, incorporating the VLP into a vaccine by addition of a pharmaceutically acceptable carrier, and administering the vaccine to the subject.
